(19) United States
(12) Patent Application Publication
(10) Pub. No.: US 2024/0139008 A1
(43) Pub. Date: May 2, 2024

(54) CATHETER AND DELIVERY SYSTEM

(71) Applicants: SB-KAWASUMI LABORATORIES, INC., Kawasaki-shi, Kanagawa (JP); SUMITOMO BAKELITE CO., LTD., Tokyo (JP)

(72) Inventors: Masako Miyahisa, Kawasaki-shi (JP); Tomoaki Yokota, Kawasaki-shi (JP)

(73) Assignees: SB-KAWASUMI LABORATORIES, INC., Kawasaki-shi, Kanagawa (JP); SUMITOMO BAKELITE CO., LTD., Tokyo (JP)

(21) Appl. No.: 18/280,772

(22) PCT Filed: Mar. 8, 2022
(86) PCT No.: PCT/JP2022/010099

§ 371 Date: Sep. 7, 2023

(30) Foreign Application Priority Data
Mar. 12, 2021 (JP) 2021-040408

(51) Int. Cl. A61F 2/966; A61F 2/844

(52) U.S. Cl. CPC A61F 2/966; A61F 2/844; A61F 2002/9665

(57) ABSTRACT

A tubular indwelling device is accurately placed at a placement target site in a biological lumen. A catheter (1) is a catheter for delivering a colonic stent (2) to be placed in a biological lumen, including: a sheath (11); an inner tube (12) configured to be movable in the sheath in an axial direction; and converting means (20) capable of maintaining at least a portion of the colonic stent in the axial direction in a contracted state in a state in which the colonic stent is released from the sheath and converting the portion from the contracted state to an expanded state. The inner tube is provided with a guide rib (30) that holds the converting means, and the guide rib is formed to be able to position the colonic stent in the contracted state in an axial direction of the inner tube.

### TECHNICAL FIELD

The present invention relates to a catheter and a delivery system.

### BACKGROUND ART

In the related art, a tubular indwelling device (generally called as a “stent”) that is placed in a narrowed part or an occluded part occurring in the digestive tract such as the esophagus, stomach, small intestine, large intestine, and bile duct or in a blood vessel (hereinafter, referred to as a “biological lumen”) and expands a lesion site to maintain the patency of the biological lumen has been known (for example, refer to Patent Document 1). The tubular indwelling device is delivered to a placement target site using a catheter.

### PRIOR ART DOCUMENT

### Patent Document

[Patent Document 1] Japanese Patent No. 4651943

### SUMMARY OF INVENTION

### Technical Problem

Here, the tubular indwelling device is attached to a shaft-like member of the catheter in a radially contracted state and accommodated in a sheath. However, there is concern that when the tubular indwelling device is released from the sheath in a state in which a portion of the tubular indwelling device in an axial direction is contracted, an outer surface of the tubular indwelling device comes into contact with an inner surface of the sheath, and the tubular indwelling device is displaced with respect to the shaft-like member in an axial direction thereof. In addition, there is concern that the catheter is bent depending on, for example, a direction of a tension generated when pulling out a string-like member for maintaining the tubular indwelling device in the contracted state, resulting in not only the displacement of the tubular indwelling device in the axial direction of the shaft-like member but also deformation of a shape. As a result, it becomes difficult to accurately place the tubular indwelling device at the placement target site in the biological lumen.

An object of the present invention is to provide a catheter and a delivery system capable of accurately placing a tubular indwelling device at a placement target site in a biological lumen.

### Solution to Problem

A catheter according to the present invention is a catheter for delivering a tubular indwelling device to be placed in a biological lumen, including: a sheath; a long shaft-like member configured to be movable in the sheath in an axial direction; and converting means capable of maintaining at least a portion of the tubular indwelling device in the axial direction in a contracted state in a state in which the tubular indwelling device is released from the sheath and converting the portion from the contracted state to an expanded state, in which the shaft-like member is provided with a holding portion that holds the converting means, and the holding portion is formed to be able to position the tubular indwelling device in the contracted state in an axial direction of the shaft-like member.

A delivery system according to the present invention includes: a tubular indwelling device to be placed in a biological lumen; and a catheter for delivering the tubular indwelling device into a biological lumen, in which the catheter includes a sheath, a long shaft-like member configured to be movable in the sheath in an axial direction, and converting means capable of maintaining at least a portion of the tubular indwelling device in the axial direction in a contracted state in a state in which the tubular indwelling device is released from the sheath and converting the portion from the contracted state to an expanded state, the shaft-like member is provided with a holding portion that holds the converting means, and the holding portion is formed to be able to position the tubular indwelling device in the contracted state in an axial direction of the shaft-like member.

### Advantageous Effects of Invention

According to the present invention, a tubular indwelling device can be accurately placed at a placement target site in a biological lumen.

### BRIEF DESCRIPTION OF DRAWINGS

FIGS. 1A and 1B are views showing a configuration of a delivery system.

FIG. 2 is a schematic view showing an example of converting means.

FIGS. 3A and 3B are schematic views showing a portion of the delivery system to which a colonic stent is attached.

FIG. 4 is a schematic view showing an example of a guide rib.

FIGS. 5A to 5C are schematic views showing an example of a change in a state of the colonic stent during placement.

FIGS. 6A and 6B are schematic views showing an example of the guide rib.

FIGS. 7A to 7C are cross-sectional views schematically showing another example of the guide rib.

### DESCRIPTION OF EMBODIMENTS

Hereinafter, an embodiment of the present invention will be described in detail with reference to the drawings. In the present embodiment, as an example of the present invention, a catheter 1 and a delivery system 100 for placing a colonic stent 2, which is a tubular indwelling device, in the large intestine C to cause a lesion site L (for example, a narrowed part or an occluded part of the large intestine) of the large intestine C to expand radially outward and treat an occlusion (stenosis) will be described.

A delivery device for placing the colonic stent 2 at a placement target site is referred to as “catheter 1”, and a system in which the catheter 1 is loaded with the colonic stent 2 is referred to as “delivery system 100”.

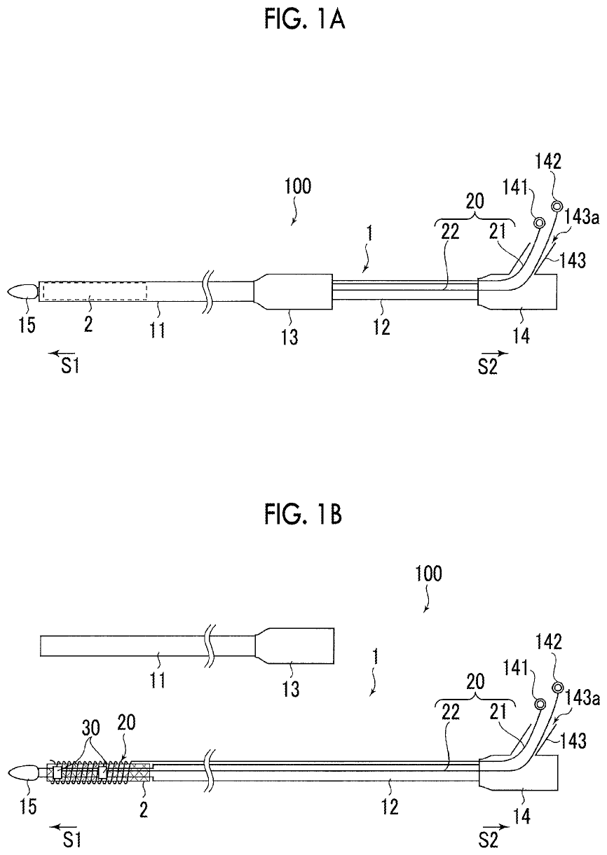

FIGS. 1A and 1B are views showing a configuration of the delivery system 100. FIG. 1A shows the delivery system 100 in an assembled state, and FIG. 1B shows the delivery system 100 in a disassembled state. FIGS. 1A and 1B schematically show a size (length, diameter, and the like) and a shape of each member constituting the delivery system 100 in order to facilitate understanding of the invention. In the following description, a left side in FIGS. 1A and 1B is a distal side S1, and a right side is a proximal side S2.

When the colonic stent 2 is placed in the large intestine, the delivery system 100 is inserted into a forceps hole of an endoscope and is endoscopically used.

As shown in FIGS. 1A and 1B, the delivery system 100 includes the catheter 1 and the colonic stent 2.

The colonic stent 2 has a tubular shape that defines a tubular flow channel through which the digested material flows. The colonic stent 2 is, for example, a bare stent composed only of a skeleton. The skeleton is a reinforcing member for maintaining an expanded state of the colonic stent 2, and is formed to be self-expandable from an inwardly contracted state to an outwardly expanded state in a radial direction substantially perpendicular to the axial direction.

The skeleton is formed, for example, by weaving wires into a tubular shape. For example, the skeleton is formed by weaving two wires that extend spirally while being folded back in a zigzag shape (Z shape) at a predetermined pitch into a rhombic wire mesh shape (fence shape) in which bent portions (peak portions on one side (portions protruding toward one end side in the axial direction) and valley portions on the other side (portions protruding toward the other end side in the axial direction)) mesh with each other. In this case, in a state in which tension is applied to the colonic stent 2 in the axial direction, the bent portions of the wires forming the mesh intersect each other densely, so that stretching of the colonic stent 2 in the axial direction is restricted.

The skeleton is not limited to the above-mentioned skeleton structure. For example, the skeleton may be a spiral skeleton configured by spirally winding one metal wire while bending the metal wire in a zigzag shape (Z shape) so that peak portions (bent portions on a tip end side) and valley portions (bent portions on a rear end side) are alternately formed. Alternatively, the skeleton may be formed by subjecting a metallic cylindrical member to laser processing. Furthermore, a plurality of wires may be used to form the skeleton.

Examples of a material forming the skeleton include known metals or metal alloys represented by stainless steel, a nickel-titanium alloy (nitinol), and a titanium alloy. Also, an alloy material having X-ray contrast properties may be used. In this case, a position of the colonic stent 2 can be checked from outside the body. The skeleton may be formed of a material other than metal (for example, ceramic or a resin).

The material of the wire forming the skeleton, a wire type (for example, a circular wire such as wire or a rectangular wire obtained by laser cutting), a cross-sectional area (corresponding to a wire diameter in a case of a round wire), the number of bends and a bend shape in a circumferential direction (the number of peak portions and a shape of the peak portion), a wire interval in the axial direction (the amount of skeleton per unit length), and the like can be selected as appropriate based on storability required for the colonic stent 2 in the sheath depending on a placement site, releasability from the sheath, placeability (corresponding to an expansion force), flexibility (bendability), and the like.

The catheter 1 includes a sheath 11, an inner tube 12, a first operating portion 13, a second operating portion 14, a distal tip 15, converting means 20, guide ribs 30, and the like.

The sheath 11 is, for example, a tubular member formed of a flexible material. The inner tube 12 is, for example, a long shaft-like member made of a material having appropriate hardness and flexibility, such as a resin or metal.

Proximal sides S2 of the sheath 11 and the inner tube 12 are respectively connected to the first operating portion 13 and the second operating portion 14. The distal tip 15 is disposed at a distal end of the inner tube 12.

The inner tube 12 is inserted into the sheath 11 and is disposed to extend to a distal side S1 of the sheath 11. The sheath 11 and the inner tube 12 are configured to be relatively movable in an axial direction of the sheath 11 by operating the first operating portion 13 and the second operating portion 14.

Although not shown, in the inner tube 12 and the distal tip 15, for example, a lumen through which a guide wire passes, and a lumen through which the converting means 20 for expanding the colonic stent 2 in the contracted state at an affected area passes, and the like may be formed along the axial direction.

The colonic stent 2 is attached to an end portion of the inner tube 12 on the distal side S1 and is accommodated in the sheath 11 in a radially expandable contracted state. Specifically, the colonic stent 2 is subjected to the contracted state by being folded in the radial direction while being stretched in the axial direction, and is accommodated in the sheath 11. At this time, the colonic stent 2 is held in the contracted state by the converting means 20.

The converting means 20 has a configuration capable of maintaining at least a portion (for example, a first stent portion 2A) of the colonic stent 2 in the axial direction in a contracted state in a state in which the colonic stent 2 is released from the sheath 11 and converting the portion from the contracted state to the expanded state.

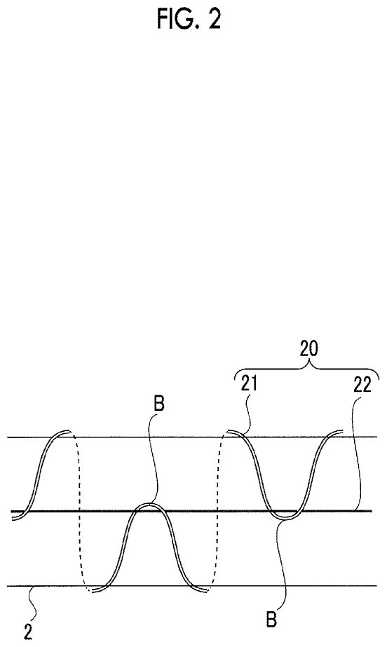

An example of the converting means 20 is shown in FIG. 2. The converting means 20 includes a restraining string 21 and a holding wire 22, which are linear members. Note that a configuration other than the linear member may be applied to the converting means 20.

As shown in FIG. 2, the converting means 20 includes the restraining string 21 wound around an outer peripheral surface of the colonic stent 2 and the holding wire 22 engaged with the restraining string 21. For example, the converting means 20 is attached to the colonic stent 2 on the inner tube 12 so as to contract the colonic stent 2 in the radial direction thereof.

The restraining string 21 and the holding wire 22 are formed of, for example, a material having predetermined strength and rigidity, and for example, a suture such as nylon fibers and fluorine fibers, a nickel-titanium alloy, a thin metal wire made of stainless steel, and a string-like member made of a resin can be applied. The restraining string 21 and the holding wire 22 are preferably formed of different materials in order to improve slipperiness when pulled out.

The colonic stent 2 is held in the contracted state by portions of the restraining string 21 and the holding wire 22 on the distal side S1. For example, end portions of the restraining string 21 and the holding wire 22 on the proximal side S2 are pulled out from one branch port 143a of a Y connector 143 of the second operating portion 14 and are respectively connected to pull-out operating portions 141 and 142. By pulling the pull-out operating portions 141 and 142 in a predetermined direction (for example, an upper right direction in FIG. 1A), the restraining string 21 and the holding wire 22 can be pulled out. Alternatively, a dial (not shown) may be provided in the second operating portion 14, and the holding wire 22 may be pulled out while performing fine adjustment by rotating the dial.

In the example shown in FIG. 2, the restraining string 21 is wound in the circumferential direction around an outer peripheral surface of the portion of the colonic stent 2 (for example, the first stent portion 2A), and is bent and wound in an opposite direction for each rotation. On the other hand, the holding wire 22 is disposed along an axial direction of the colonic stent 2 and is engaged with a bent portion B formed in the restraining string 21. That is, the restraining string 21 is wound in such a manner that the restraining string 21 cannot hold the wound state by itself, and is held so as not to fall off by being engaged with the holding wire 22. Therefore, when the engagement between the restraining string 21 and the holding wire 22 is released, the restraining string 21 naturally falls off the colonic stent 2. As a result, the portion of the colonic stent 2 is released from the contracted state and transitions to the expanded state.

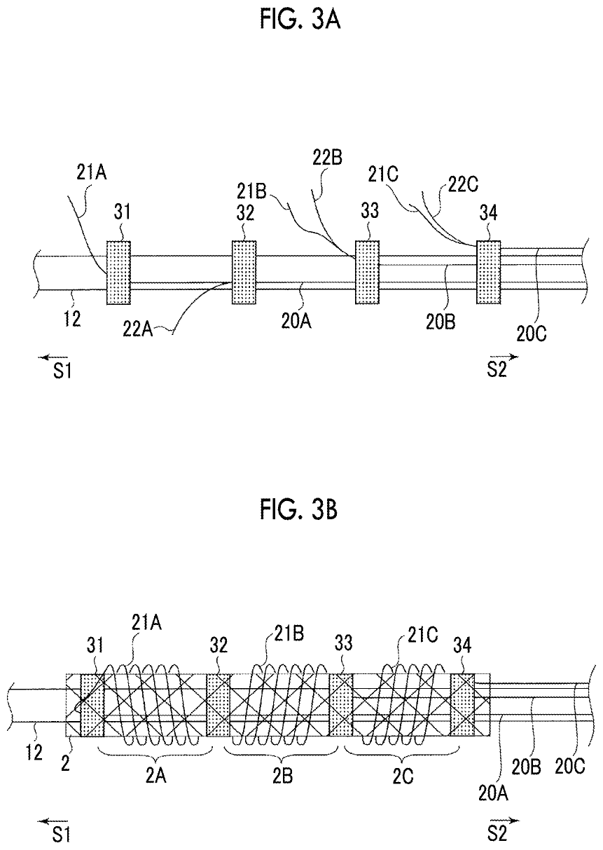

First to third converting means 20A to 20C are provided corresponding to first to third stent portions 2A to 2C, which divide the colonic stent 2 into three sections in the axial direction (see FIGS. 3A and 3B). The first stent portion 2A is a portion positioned on the most distal side S1, the third stent portion 2C is a portion positioned on the most proximal side S2, and the second stent portion 2B is a portion between the first stent portion 2A and the second stent portion 2B.

As shown in FIGS. 3A and 3B, the first to third converting means 20A to 20C have first to third restraining strings 21A to 21C and first to third holding wires 22A to 22C, respectively. Note that the first to third holding wires 22A to 22C are omitted in FIG. 3B. The first to third converting means 20A to 20C are configured to be independently operable, and expansion of a stent portion (for example, the first stent portion 2A) to be expanded among the first stent portion 2A to the third stent portion 2C of the colonic stent 2 can be flexibly controlled while checking a placement state and the like.

The guide ribs 30 are disposed on an outer peripheral surface of a stent attachment portion, which is the end portion of the inner tube 12 on the distal side S1. The guide ribs 30 are fixed to the inner tube 12 by, for example, adhesion or welding. In addition, the guide ribs 30 are formed to be able to position the colonic stent 2 in the contracted state in an axial direction of the inner tube 12. For example, in a state in which the colonic stent 2 is attached to the inner tube 12, the skeleton of the colonic stent 2 is hooked on an end face of the guide rib 30 and is thus positioned in the axial direction of the inner tube 12, so that the colonic stent 2 can be moved in conjunction with the inner tube 12. Furthermore, even when the holding wire 22 is pulled out, the skeleton of the colonic stent 2 in the contracted state is hooked on the end face of the guide rib 30, so that the colonic stent 2 can be expanded without axial displacement of the colonic stent 2 with respect to the inner tube 12.

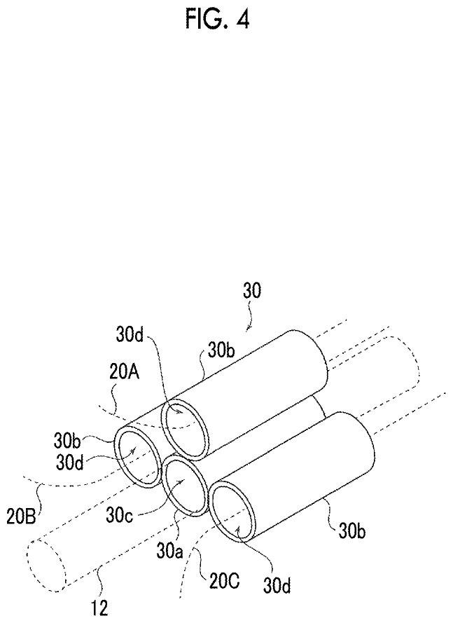

An example of the guide rib 30 is shown in FIG. 4. As shown in FIG. 4, the guide rib 30 has a tube fixing portion 30a and engagement portions 30b. The guide rib 30 is formed, for example, by welding four cylindrical members. In the guide rib 30, three engagement portions 30b are disposed at, for example, 90° intervals on a peripheral surface of the tube fixing portion 30a.

The tube fixing portion 30a has, for example, a cylindrical shape. The inner tube 12 is inserted through a through-hole 30c provided in the tube fixing portion 30a. That is, the through-hole 30c functions as an attachment portion to which the inner tube 12 is attached (hereinafter referred to as “attachment portion 30c”).

The engagement portion 30b has a shape that can be engaged with the colonic stent 2 in the contracted state. The engagement portion 30b has, for example, a cylindrical shape like the tube fixing portion 30a. The converting means 20 is inserted through a through-hole 30d provided in the engagement portion 30b. That is, the through-hole 30d functions as a guide portion (hereinafter, referred to as “guide portion 30d”) through which the converting means 20 (restraining string 21 and holding wire 22) is inserted to hold the converting means 20.

In the guide rib 30 shown in FIG. 4, sizes (outer diameter, length) and shapes of the tube fixing portion 30a and the engagement portion 30b are the same, but may also be different from each other. A maximum outer diameter of the guide rib 30, that is, an outer diameter of a circle circumscribing the guide rib 30 is set to be smaller than an inner diameter of the sheath 11 even in a state in which the colonic stent 2 is placed on a peripheral surface of the guide rib 30.

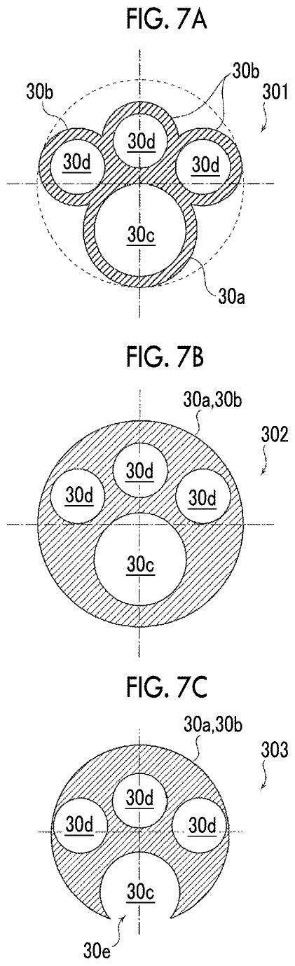

Moreover, the shape of the guide rib 30 is merely an example and is not limited thereto. The shape of the guide rib 30 can be changed as appropriate. For example, the guide rib may be formed of a triple-lumen tube which is a molded component, and the converting means 20 may be inserted through a plurality of lumens. Alternatively, the guide rib may be formed into a protruding shape like a rib and a protruding portion thereof may be provided with the guide portion 30d through which the converting means 20 is inserted (see FIGS. 7A to 7C).

The guide rib 30 shown in FIG. 4 is provided with three guide portions 30d, and the first to third converting means 20A to 20C can be individually inserted through the guide portions 30d to be guided. Accordingly, it is possible to prevent the first to third converting means 20A to 20C from being entangled with each other, and to reduce resistance when the first to third converting means 20A to 20C are pulled out. Therefore, among the first to third holding wires 22A to 22C, only the desired holding wire (for example, the first holding wire 22A) can be smoothly pulled out. In addition, among the first to third restraining strings 21A to 21C, only the desired restraining string (for example, the first restraining string 21A) can be smoothly pulled out.

In addition, the engagement portion 30b is provided to protrude from the tube fixing portion 30a, so that the colonic stent 2 can be engaged with the engagement portion 30b during stent attachment. Accordingly, when the colonic stent 2 is placed, particularly, for example, when the colonic stent 2 is released from the sheath 11, an engaged state between the engagement portion 30b and the colonic stent 2 is held, and the colonic stent 2 is less likely to be displaced with respect to the inner tube 12.

In addition, the guide rib 30 has a cross-sectional shape that partially protrudes radially outward. That is, the attachment portion 30c is provided eccentrically with respect to the circumscribed circle of the guide rib 30 in a cross section perpendicular to the axial direction. Accordingly, an effect on a filling rate in the sheath 11 is reduced, and the releasability when the colonic stent 2 is released from the sheath 11 is improved.

In the present embodiment, the catheter 1 includes first to fourth guide ribs 31 to 34 as the guide ribs 30. The first to fourth guide ribs 31 to 34 are disposed along the axial direction of the inner tube 12 so as to be spaced in the axial direction in order from the distal side S1 (see FIGS. 3A and 3B). The first stent portion 2A of the colonic stent 2 is positioned between the first and second guide ribs 31, 32, the second stent portion 2B is positioned between the second and third guide ribs 32 and 33, and the third stent portion 2C is positioned between the third and fourth guide ribs 33 and 34.

The first converting means 20A is inserted through the fourth guide rib 34, the third guide rib 33, and the second guide rib 32 in this order from the proximal side S2. The first holding wire 22A of the first converting means 20A is inserted through the second guide rib 32, is then pulled out from the colonic stent 2, and is disposed along the first stent portion 2A. The first restraining string 21A is also inserted through the first guide rib 31, is folded back, and is then wound around an outer peripheral surface of the first stent portion 2A from the distal side S1 toward the proximal side S2. The first restraining string 21A does not necessarily have to be inserted through the first guide rib 31, and may only have a function of causing the colonic stent 2 to be engaged with the first guide rib 31.

The second converting means 20B is inserted through the fourth guide rib 34 and the third guide rib 33 in this order from the proximal side S2. The second holding wire 22B of the second converting means 20B is inserted through the third guide rib 33, is then pulled out from the colonic stent 2, and is disposed along the second stent portion 2B. Similarly to the second holding wire 22B, the second restraining string 21B is inserted through the third guide rib 33, is then pulled out from the colonic stent 2, and is wound around an outer peripheral surface of the second stent portion 2B from the proximal side S2 toward the distal side S1.

The third converting means 20C is inserted only through the fourth guide rib 34 on the most proximal side S2. The third holding wire 22C of the third converting means 20C is inserted through the fourth guide rib 34, is then pulled out from the colonic stent 2, and is disposed along the third stent portion 2C. Similarly to the third holding wire 22C, the third restraining string 21C is inserted through the fourth guide rib 34, is then pulled out from the colonic stent 2, and is wound around an outer peripheral surface of the third stent portion 2C from the proximal side S2 toward the distal side S1. The third converting means 20C does not necessarily have to be inserted through the fourth guide rib 34, and may only have a function of suppressing bending of the inner tube 12 and the catheter 1, which will be described later, since the first and second converting means 20A and 20B are inserted through the fourth guide rib 34.

As described above, a plurality of first to third converting means 20A to 20C are provided corresponding to the second to fourth guide ribs 32 to 34. Accordingly, by appropriately pulling out the first to third converting means 20A to 20C that have passed through the second to fourth guide ribs 32 to 34 from the colonic stent 2, the first to third stent portions 2A to 2C that have to be restrained by the first to third converting means 20A to 20C can be distinguished and easily restrained.

In addition, in a case where the colonic stent 2 is to be placed at the placement target site, the first to third holding wires 22A to 22C can be pulled out along the axial direction of the inner tube 12, and bending of the inner tube 12 and the catheter 1 can be suppressed.

From the viewpoint of productivity, the first to fourth guide ribs 31 to 34 may be made of members having the same shape and material. However, in order to improve the releasability from the sheath 11, it is preferable that the guide portions 30d are provided according to the number of first to third converting means 20A to 20C that have passed through the guide rib 30. That is, for example, the fourth guide rib 34 may be provided with three guide portions 30d corresponding to the first to third converting means 20A to 20C, the third guide rib 33 may be provided with two guide portions 30d corresponding to the first and second converting means 20A and 20B, and the second guide rib 32 may be provided with one guide portion 30d corresponding to the first converting means 20A.

In addition, in a case where an interval between the guide ribs 30 is wide, it is difficult to pull out the holding wire 22 when the placement site of the colonic stent 2 is curved, and a large tension is applied to the holding wire 22, which may cause the holding wire 22 to rub against and damage the placement site. Therefore, the number of guide ribs 30 may be made larger than the number of converting means 20 so that the interval between the guide ribs 30 is narrowed. Accordingly, it becomes difficult for the holding wire 22 to be separated from the inner tube 12 when the holding wire 22 is pulled out, thereby preventing the holding wire 22 from rubbing against and damaging the placement site.

Moreover, it is preferable that an end face of the first guide rib 31 where the first restraining string 21A is folded back is chamfered. Accordingly, it is possible to reduce kinks and damages that occur in the first restraining string 21A due to rubbing against the first guide ribs 31 in a case where the first restraining string 21A folded back toward the proximal side S2 is recovered. Furthermore, the first guide rib 31 may have a structure capable of accommodating an excess first restraining string 21A.

In addition, the first guide rib 31 on the most distal side S1 does not necessarily need to be disposed, and the first restraining string 21A may be inserted through the second guide rib 32, then pulled out from the colonic stent 2, and wound around the outer peripheral surface of the first stent portion 2A from the proximal side S2 towards the distal side S1. In a case where the first guide rib 31 is disposed, the guide portion 30d may be a bottomed hole for holding a tip end of the holding wire 22 instead of the through-hole.

The guide rib 30 is formed, for example, by injection molding in which a molten resin is injected into a mold and cooled and solidified. Alternatively, for example, the guide rib 30 may be formed by extrusion or cutting of metal components, or by three-dimensional modeling. In addition, a raw material resin may contain a material that enhances contrast properties.

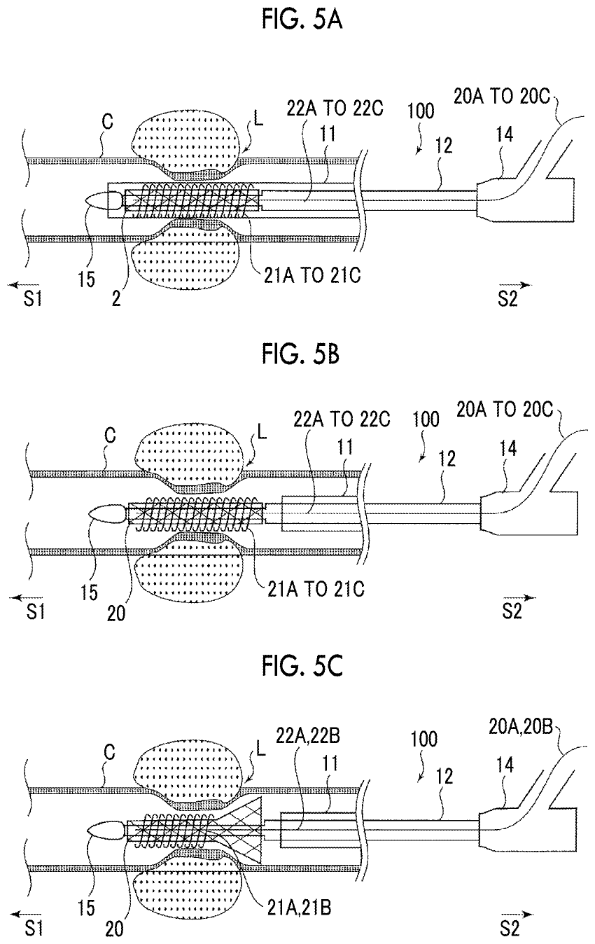

FIGS. 5A to 5C are views showing a change in a state of the colonic stent 2 during placement. FIGS. 5A to 5C schematically show the colonic stent 2, and illustration of detailed configurations of the first to third converting means 20A to 20C is omitted. In FIGS. 5A to 5C, the distal side S1 is an “oral side” and the proximal side S2 is an “anal side”. Here, a case will be described in which the colonic stent 2 is placed by expanding from the third stent portion 2C on the proximal side S2.

In a case where the colonic stent 2 is to be placed at the lesion site L (placement target site) of the large intestine C, the sheath 11 and the inner tube 12 are inserted from the anal side along the guide wire (not shown) previously introduced into the large intestine C and are positioned so that the colonic stent 2 is positioned at the lesion site L (see FIG. 5A).

Next, in the positioned state, the sheath 11 is moved to the proximal side S2 (anal side) to release the colonic stent 2 from the sheath 11 (see FIG. 5B). At this time, the colonic stent 2 is restrained by the first to third converting means 20A to 20C held by the guide ribs 30 (first to fourth guide ribs 31 to 34) and maintained in the contracted state. In addition, the colonic stent 2 is released from the sheath 11 in a state in which axial displacement of the colonic stent 2 with respect to the inner tube 12 is restricted by the guide ribs 30 (first to fourth guide ribs 31 to 34).

The colonic stent 2 may be released from the sheath 11 by moving the inner tube 12 so as to be extruded toward the oral side in a state in which a position of the sheath 11 is fixed.

When the entire colonic stent is expanded as the colonic stent is released from a sheath as in the related art, the colonic stent is positioned by an expansion force thereof, making it difficult to adjust a placement position. In addition, since a placement state can be endoscopically checked at the time when the colonic stent is expanded from the distal side S1 to the proximal side S2, it is difficult to adjust the placement position during placement. Contrary to this, in the present embodiment, since the colonic stent 2 does not expand at the time when the colonic stent 2 is released from the sheath 11, the placement position of the colonic stent 2 can be adjusted while endoscopically checking the position on the proximal side S2 in the state shown in FIG. 5B. Therefore, the colonic stent 2 can be easily placed at an appropriate position. Also, the colonic stent 2 can be accommodated again in the sheath 11.

Next, for example, the third holding wire 22C is pulled out based on the operation of the pull-out operating portion 142 of the second operating portion 14 to release the restraint of the third stent portion 2C of the colonic stent 2 by the third converting means 20C and release the third stent portion 2C. Accordingly, only the third stent portion 2C transitions to the expanded state (see FIG. 5C). At this time, the first stent portion 2A and the second stent portion 2B are maintained in the contracted state by the first and second converting means 20A and 20B. The third restraining string 21C is appropriately pulled out and recovered.

Similarly, the first stent portion 2A and the second stent portion 2B of the colonic stent 2 are released and enter the expanded state, and the colonic stent 2 is properly placed at the target placement position.

As described above, the catheter 1 according to the present embodiment is the catheter 1 for delivering the colonic stent 2 (tubular indwelling device) to be placed in the large intestine C (biological lumen), and includes the sheath 11, the inner tube 12 (shaft-like member) configured to be movable in the sheath 11 in the axial direction, and the converting means 20 capable of maintaining at least a portion (for example, the first stent portion 2A) of the colonic stent 2 in an axial direction in a contracted state in a state in which the colonic stent 2 is released from the sheath 11 and converting the portion from the contracted state to an expanded state. In addition, the inner tube 12 is provided with the guide ribs 30 (holding portion) for holding the converting means 20, and the guide ribs 30 are formed to be able to position the colonic stent 2 in the contracted state in the axial direction of the inner tube 12.

According to the catheter 1, when the colonic stent 2 is released from the sheath 11, holding of the converting means 20 and positioning of the colonic stent 2, in which at least a portion is in a contracted state, in the axial direction with respect to the inner tube 12 can be properly performed by the guide ribs 30, so that delivery of the colonic stent 2 can be properly performed. In addition, the colonic stent 2 can be partially and properly expanded in the axial direction in various manners according to an introduction direction of the catheter 1, the placement site, and the like. At this time, for example, the holding wire 22 as the converting means 20 can be pulled out along the inner tube 12 by the guide ribs 30, so that bending of the inner tube 12 and the catheter 1 can be suppressed. As a result, the colonic stent 2 can be accurately placed at the placement target site in the large intestine C.

In addition, in the catheter 1, a plurality of the guide ribs 30 (holding portions) are provided spaced apart in the axial direction of the inner tube 12 (shaft-like member), and a plurality of the converting means 20 (first to third converting means 20A to 20C) are provided corresponding to the plurality of guide ribs 30 (second to fourth guide ribs 32 to 34).

Accordingly, even in a configuration in which a plurality of portions of the colonic stent 2 can be partially expanded by the plurality of converting means 20, the plurality of converting means 20 can be properly held by the plurality of guide ribs 30. In addition, by the plurality of guide ribs 30, positioning of the colonic stent 2 with respect to the inner tube 12 in the axial direction thereof can be more properly performed.

In addition, in the catheter 1, the guide rib 30 (holding portion) has the engagement portion 30b that can be engaged with the colonic stent 2 (tubular indwelling device) in the contracted state.

Accordingly, the engagement portion 30b and the colonic stent 2 are engaged with each other in a state in which the colonic stent 2 is attached to the inner tube 12. Therefore, the colonic stent 2 is less likely to be displaced with respect to the inner tube 12, and the colonic stent 2 in which at least a portion is in a contracted state can be easily positioned with respect to the inner tube 12 in the axial direction thereof by the guide ribs 30.

In addition, in the catheter 1, the converting means 20 has the restraining string 21 and the holding wire 22 (at least one linear member), and the guide rib 30 (holding portion) has the attachment portion 30c to which the inner tube 12 (shaft-like member) is attached and the guide portion 30d through which the restraining string 21 and the holding wire 22 are inserted.

Accordingly, a portion of the colonic stent 2 can be converted from the contracted state to the expanded state by a simple operation of pulling out the restraining string 21 and the holding wire 22.

In addition, the attachment portion 30c is provided eccentrically with respect to the circumscribed circle of the guide rib 30 in a cross section perpendicular to the axial direction.

Accordingly, the effect on the filling rate in the sheath 11 is reduced, and the releasability when the colonic stent 2 is released from the sheath 11 is improved.

In addition, the guide portion 30d is provided in the engagement portion 30b. That is, the engagement portion 30b is configured so that the restraining string 21 and the holding wire 22 can be inserted therethrough.

Accordingly, not only can the restraining string 21 and the holding wire 22 be properly guided by the guide portion 30d, but also the guide rib 30 and the colonic stent 2 can be engaged with each other using the engagement portion 30b. Therefore, the configuration of the guide rib 30 can be simplified.

Although the invention made by the inventor of the present invention has been specifically described above based on the embodiments, the present invention is not limited to the above embodiments, and can be changed without departing from the scope of the invention.

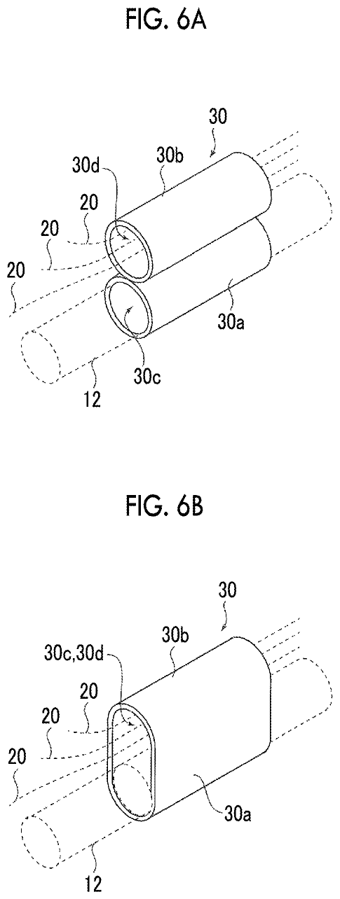

For example, the structure of the guide rib 30 shown in the embodiment is an example, and the present invention is not limited thereto. For example, as shown in FIG. 6A, a plurality of converting means 20 may be inserted through one guide portion 30d, or as shown in FIG. 6B, a plurality of converting means 20 may be inserted through a portion other than a portion through which the inner tube 12 is inserted and one through-hole is shared by the attachment portion 30c and the guide portion 30d. Moreover, an engagement portion provided in the guide rib 30 to be engaged with the colonic stent 2 may be provided separately from the engagement portion 30b, or may be formed on the peripheral surface of the guide rib 30 in a recessed shape.

FIGS. 7A to 7C are cross-sectional views schematically showing another example of the guide rib 30. In FIGS. 7A to 7C, guide ribs 301 to 303 are shown in a cross section perpendicular to the axial direction. The guide ribs 301 to 303 are different from the guide rib 30 (see FIG. 4) of the embodiment in that the guide ribs 301 to 303 are formed of a molded product made of one member.

The guide rib 301 shown in FIG. 7A has the tube fixing portion 30a and the engagement portions 30b. The three engagement portions 30b are connected to each other and provided on the peripheral surface of the tube fixing portion 30a so as to protrude from the tube fixing portion 30a. That is, the attachment portion 30c provided in the tube fixing portion 30a is provided eccentrically with respect to a circumscribed circle of the guide rib 301 (broken line in FIG. 7A).

The guide rib 302 shown in FIG. 7B has, for example, a disc shape, and through-holes are formed therein that function as the attachment portion 30c and the guide portions 30d. In the guide rib 302, it can be said that the entire guide rib 302 forms the tube fixing portion 30a, and an end face thereof in the axial direction forms the engagement portion 30b. The attachment portion 30c is provided eccentrically with respect to a circumscribed circle of the guide rib 302 (outer shape of the guide rib 302), and formation regions of the guide portions 30d are intentionally biased.

The guide rib 303 shown in FIG. 7C has, for example, a disc shape, and through-holes are formed therein that function as the attachment portion 30c and the guide portions 30d. The attachment portion 30c is not a complete “hole” that is separate from the outside. Specifically, the attachment portion 30c has an arc-shaped cross section and is formed by a groove-like notch in an outer peripheral surface of the guide rib 303. That is, the attachment portion 30c is provided eccentrically with respect to a circumscribed circle of the guide rib 303, and formation regions of the guide portions 30d are intentionally biased.

An arc shape of the attachment portion 30c is a shape that follows an outer shape of the inner tube 12. In addition, in order to prevent the inner tube 12 from coming off, a central angle of the arc shape is preferably, for example, 180° or more (that is, larger than a semi-circular arc).

The guide ribs 301 to 303 shown in FIGS. 7A to 7C have the following features in addition to the features of the guide rib 30 (see FIGS. 4 and 6A) described in the embodiment.

That is, while the guide rib 30 according to the embodiment is formed by bonding or welding a plurality of cylindrical members together, the guide ribs 301 to 303 shown in FIGS. 7A to 7C are formed of a molded product made of one member.

Accordingly, the guide ribs 301 to 303 can be easily manufactured, and the tube fixing portion 30a and the engagement portion 30b cannot be separated from each other, thereby improving handling and safety. In particular, in the guide ribs 302 and 303, in a case where the attachment portion 30c is formed eccentrically, the formation regions of the guide portions 30d are formed to be biased, so that the guide portions 30d can be formed easily. An eccentricity indicating the degree of eccentricity of the attachment portion 30c is, for example, preferably 1.0 to 3.5, and more preferably 1.5 to 2.5. Here, the eccentricity is expressed by a maximum length from the attachment portion 30c to an outer peripheral edge of the guide ribs 302 and 303 in the radial direction of the attachment portion 30c/(radius of the guide ribs 302 and 303−radius of the attachment portion 30c).

Furthermore, in the guide rib 303 shown in FIG. 7C, a notch 30e is provided in a part of the attachment portion 30c. Accordingly, the guide rib 303 in FIG. 7C has a smaller outer diameter than the guide rib 302 in FIG. 7B in a case where the area of the formation region of the guide portion 30d is set to be about the same, so that a reduction in size of the guide rib 303 can be achieved. Therefore, the effect on the filling rate in the sheath 11 is further reduced, and the releasability when the colonic stent 2 is released from the sheath 11 is improved. In addition, the guide rib 303 of FIG. 7C has a wider formation region of the guide portion 30d than the guide rib 302 of FIG. 7B in a case where the outer diameter is the same, so that the formation of the guide portion 30d is further facilitated.

Although FIGS. 7A to 7C show the eccentric type guide ribs 301 to 303, the guide rib 30 may be a concentric type in which the attachment portion 30c is provided concentric with the circumscribed circle of the guide rib 30 in the cross section perpendicular to the axial direction. In this case, regardless of a shape of the lesion site L, the engagement between the engagement portion 30b of the guide rib 30 and the colonic stent 2 is held. Therefore, axial displacement of the colonic stent 2 with respect to the inner tube 12 when the colonic stent 2 is released from the sheath 11 can be prevented.

In addition, for example, in the above embodiment, the first to third converting means 20A to 20C having independent configurations are provided, but this is merely an example and is not limited thereto. The restraining string 21 and the holding wire 22 may also be shared. For example, shapes and lengths of the restraining string 21 and the holding wire 22 may be adjusted to release the colonic stent 2 in a predetermined order according to an operation amount or an operation manner. Furthermore, the number of converting means is an example and is not limited thereto, and at least one converting means may be provided.

In addition, in the embodiment, the case in which the entire colonic stent 2 in the axial direction is maintained in the contracted state by the first to third converting means 20A to 20C has been described, but the converting means 20 may maintain at least a portion of the colonic stent 2 in a contracted state. For example, during release from the sheath 11, the contracted state of the first stent portion 2A and the second stent portion 2B of the colonic stent 2 may be maintained by the first and second converting means 20A and 20B, and the third stent portion 2C may be deployed upon release from the sheath 11.

In addition, in the embodiment, the colonic stent 2, which is a straight type bare stent, has been illustrated and described as the tubular indwelling device to be placed by the delivery system 100, but this is merely an example and is not limited thereto. For example, a single-sided or double-sided flared stent may be applied to the tubular indwelling device to be placed by the delivery system 100. Alternatively, the tubular indwelling device may be a covered stent in which a membrane is disposed entirely or partially around a peripheral surface of a skeleton. However, in the case of a covered stent, a mesh type membrane having pores is suitable in order to pull out the converting means 20 from the inside to the outside.

Furthermore, the present invention is not limited to the colonic stent 2 described in the embodiment, but may also be applied to a digestive tract stent placed in the digestive tract such as the esophagus and large intestine, and a stent graft placed in a blood vessel. In this case, examples of a fluid flowing through the digestive tract include food immediately after ingestion that has not been digested at all, a product of the breakdown of food as the food passes through the digestive tract, and a material that has not been digested after passing through the digestive tract (for example, stool), regardless of a state of the material.

The embodiments disclosed herein are merely examples in all respects, and should not be considered restrictive. The scope of the present invention is represented by the appended, not by the above description, and is intended to include all modifications within the meaning and the scope which are equivalent to those of the appended claims.

The disclosure contents of the specification, drawings and abstract contained in the Japanese application of Japanese Patent Application No. 2021-040408 filed on Mar. 12, 2021 are all incorporated herein by reference.

### REFERENCE SIGNS LIST

1: catheter
2: colonic stent (tubular indwelling device)
11: sheath
12: inner tube (shaft-like member)
20: converting means
21: restraining string (linear member)
22: holding wire (linear member)
30, 301 to 303: guide rib (holding portion)
30b: engagement portion
30c: attachment portion
30d: guide portion
100: delivery system
C: large intestine (biological lumen)

1. A catheter for delivering a tubular indwelling device to be placed in a biological lumen, comprising:
a sheath;
a long shaft-like member configured to be movable in the sheath in an axial direction; and
converting units configured to be capable of maintaining at least a portion of the tubular indwelling device in the axial direction in a contracted state in a state in which the tubular indwelling device is released from the sheath and converting the portion from the contracted state to an expanded state,
wherein the shaft-like member is provided with a holding portion that holds the converting units, and
the holding portion is formed to be able to position the tubular indwelling device in the contracted state in an axial direction of the shaft-like member.

2. The catheter according to claim 1,
wherein a plurality of the holding portions are provided spaced apart in the axial direction of the shaft-like member, and
a plurality of the converting means are provided corresponding to the plurality of holding portions.

3. The catheter according to claim 1,
wherein the holding portion has an engagement portion that is engageable with the tubular indwelling device in the contracted state.

4. The catheter according to claim 1,
wherein the converting units has at least one linear member, and
the holding portion is a guide rib having an attachment portion to which the shaft-like member is attached and a guide portion through which the linear member is inserted.

5. The catheter according to claim 4,
wherein the attachment portion is provided eccentrically with respect to a circumscribed circle of the guide rib in a cross section perpendicular to the axial direction.

6. The catheter according to claim 4,
wherein the guide rib has an engagement portion that is engageable with the tubular indwelling device in the contracted state, and
the guide portion is provided in the engagement portion.

7. The catheter according to claim 5,
wherein a notch is provided in a part of the attachment portion.

8. The catheter according to claim 4,
wherein the guide rib is a molded product made of one member.

9. A delivery system comprising:
a tubular indwelling device to be placed in a biological lumen; and
a catheter for delivering the tubular indwelling device into the biological lumen,
wherein the catheter includes
a sheath,
a long shaft-like member configured to be movable in the sheath in an axial direction, and
converting units configured to be capable of maintaining at least a portion of the tubular indwelling device in the axial direction in a contracted state in a state in which the tubular indwelling device is released from the sheath and converting the portion from the contracted state to an expanded state,
the shaft-like member is provided with a holding portion that holds the converting units, and
the holding portion is formed to be able to position the tubular indwelling device in the contracted state in an axial direction of the shaft-like member.
